(19) United States
(12) Patent Application Publication
(10) Pub. No.: US 2005/0273013 A1
(43) Pub. Date: Dec. 8, 2005

(54) Wireless patient monitoring system

(76) Inventor: Lee E. Kent, Grand Rapids, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 10/862,069

(22) Filed: Jun. 4, 2004

(51) Int. Cl. A61B 5/02

(52) U.S. Cl. 600/500; 128/903

(57) ABSTRACT

A wireless patient monitoring system generally including a communication receiver, a transmitter, a reader and optionally including a communication lead is provided. Such a wireless patient monitoring system measures a patient's biological factors, which may be monitored from a remote location, which does not produce much, if any, artifact and which may include multi-directional communication within the wireless patient monitoring system.

### BACKGROUND OF THE INVENTION

The present invention relates to a wireless patient monitoring system.

Various patient monitoring systems are known. One such monitoring system is a pulse oximetry system (hereinafter oximetry). Oximetry systems measure the arterial oxygen saturation of a patient's hemoglobin. Typically, these oximetry systems are local systems (i.e., located within a patient's room), emit a local audible alarm in the event of an abnormality, and have a uni-directional communication system which is hard-wired (i.e., not wireless).

While oximetry is a useful non-invasive way to monitor a patient's cardio-respiratory system, oximetry also has many disadvantages. The function of a pulse oximeter is affected by many variables, including, but not limited to, ambient light, shivering, abnormal hemoglobins, pulse rate and rhythms, patient temperature and various cardiac functions. Additional disadvantages include the fact that oximeters are utilized locally (i.e., within a patient's hospital room or within the patient's general immediate care area). This disadvantage becomes more evident when an audible alarm sounds to inform the caregiving staff of a potential abnormality. Often it can be difficult for the caregiving staff to determine from where these audible alarms are originating and one or more caregivers must stop what they are doing and tend to the respective alarm(s). This has essentially desensitized caregivers to pulse oximetry alarms.

Another patient monitoring system includes cardiac telemetry monitoring (i.e., electrocardiograms). Such systems measure and record, among other data, cardiac rates and cardiac rhythms. These systems are confined to cardiac telemetry beds within a healthcare institution. Essentially, telemetry systems create information that can later be evaluated and studied for diagnostic and/or prognostic purposes.

Telemetry monitoring systems have several disadvantages associated with their use. Namely, such systems are very expensive to purchase, maintain and to monitor. Additionally, because telemetry systems are very sensitive regarding the bio-information they interpret, artifact is commonly produced. Artifact is any abnormality displayed and/or recorded regarding a patient's cardiac rate and/or rhythm, which is later determined to not be attributed to an abnormality in a patient's medical state. The following are examples of activities that may create artifact: teeth brushing, coughing, and/or virtually any exertion of energy, whether it be physical energy or emotional energy, may create artifact. When such an abnormality is noticed, the immediate caregiver (i.e., typically, a nurse) must call for a physician (i.e., typically, a cardiologist) to evaluate the data produced by the telemetry system to determine whether the data warrants medical attention or whether the data is artifact, which typically does not warrant medical attention. Due to the difficulty in determining artifact retrospectively, additional needless testing is often performed. Therefore, telemetry systems in result in a great deal of time and money expended that is not warranted.

Additionally, because of the shortage of qualified caregivers in the healthcare field, telemetry systems are being used defensively. Generally, one caregiver can monitor a screen, or other display device, which displays data from multiple telemetry beds (i.e., multiple patients). This allows one caregiver to essentially oversee and monitor the status of multiple patients. Because of this shortage in qualified caregivers, many patients that may not necessarily need to be placed on telemetry beds are placed on telemetry so that one caregiver can monitor their respective status. This results in tremendous and needless cost expenditures.

Surprisingly, Applicant has developed a cost-effective and efficient wireless patient monitoring system that measures biological factors, which may be monitored from a remote location, which does not produce much, if any, artifact and which may include multi-directional communication within the wireless patient monitoring system.

### SUMMARY OF THE INVENTION

One aspect of the present invention includes a patient monitoring system. Such a system includes a stationary wireless communication receiver. The receiver receives more than one wireless communication frequency and the receiver also includes at least one display indicator. The system further includes at least one transmission device. The transmission device is in communication with a patient and is in wireless communication with the communication receiver. The transmission device includes a scanning and programmable transmitter. The transmitter interprets and wirelessly communicates a patient's biological factors to the receiver via an open wireless communication frequency. The system also includes a communication lead having a first end releasably connected to the transmitter. The communication lead includes a second end adapted to interpret a patient's biological factors. The second end is in communication with a patient.

These and other features, advantages, and objects of the present invention will be further understood and appreciated by those skilled in the art by reference to the following specification, claims, and appended drawings.

### BRIEF DESCRIPTION OF THE DRAWINGS

FIG. 1 is a representative schematic of the wireless patient monitoring system according to one aspect of the present invention;

FIG. 2 is a representative schematic of multiple transmitters wirelessly communicating with a centrally located receiver of the wireless patient monitoring system according to one aspect of the present invention;

FIG. 3 is a representative schematic of a reader, which is in wireless communication with a transmitter, wherein the transmitter is in wireless communication with a centrally located receiver of the wireless patient monitoring system according to one aspect of the present invention; and

FIG. 4 is a representative schematic of a reader 50 in wireless communication with a centrally located receiver 20 of the wireless patient monitoring system according to one aspect of the present invention.

### DETAILED DESCRIPTION OF PREFERRED EMBODIMENT

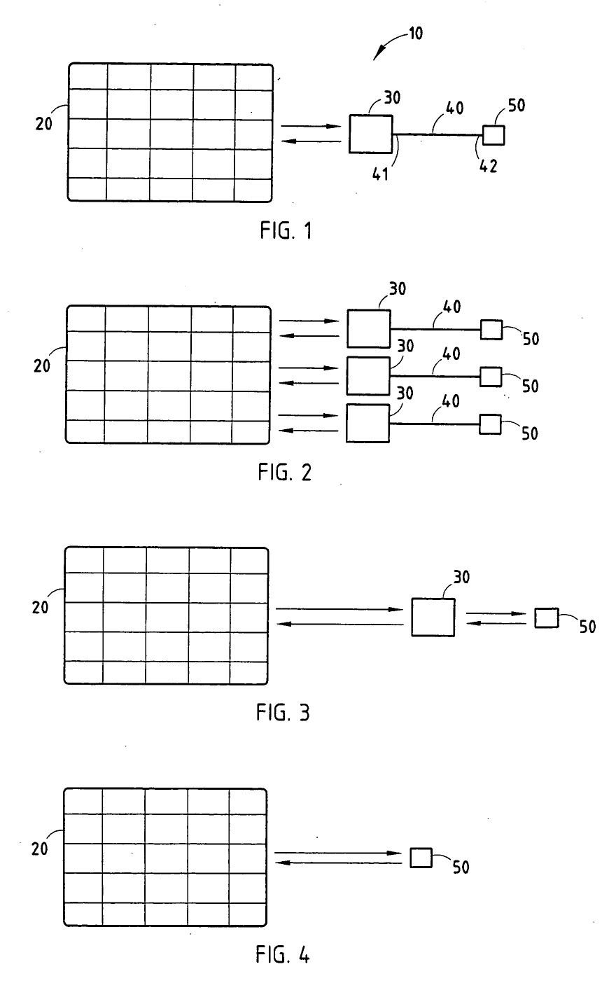

For purposes of description herein, the terms “upper,” “lower,” “right,” “left,” “rear,” “front,” “vertical,” “horizontal,” and derivatives thereof shall relate to the invention as oriented in FIG. 1. However, it is to be understood that the invention may assume various alternative orientations, except where expressly specified to the contrary. It is also to be understood that the specific devices and processes illustrated in the attached drawings and described in the following specification are exemplary embodiments of the inventive concepts defined in the appended claims. Hence, specific dimensions and other physical characteristics relating to the embodiments disclosed herein are not to be considered as limiting, unless the claims expressly state otherwise.

Referring to FIG. 1, reference numeral 10 refers to one aspect of the patient monitoring system of the present invention. Patient monitoring system 10 includes a cost-effective wireless patient monitoring system that measures a patient's biological factors, which may be monitored from a remote location, which does not produce much, if any, artifact and which may include multi-directional communication within the wireless patient monitoring system. Such a monitoring system 10 generally includes a communication receiver 20, a transmitter 30, a communication lead 40 and a reader 50.

Receiver 20 is generally a stationary wireless communication receiver of various radio frequencies. The term “wireless” as used herein refers to wireless communication. That is to say, there are no communication wires (i.e., hard-wires) that connect communication receiver 20 to transmitter 30. Therefore, the communication of information between communication receiver 20 and transmitter 30 is “wireless”. The wireless communication described and referred to herein is generally that type of wireless communication that is generally known in the art. This type of communication is typically transmitted over radio frequencies which may vary in frequency range. The range in which the present invention may effectively communicate within itself, includes, but is not limited to, a range of from about 0.5 kilohertz (kHz) to about 300,000 megahertz (mHz). The frequency ranges can be readily adjusted to accommodate the setting in which the present invention is to be used.

Communication receiver 20 is typically stationary. Communication receiver 20 is generally placed in a central location, relative to the position of transmitters 30 (i.e., at a nurses'station or the like). Communication receiver 20 may be programmed to receive any frequency, or multiple frequencies, described above within the radio frequency ranges provided. However, more typically, communication receiver 20 is programmed to receive radio frequency ranges within the range of from about 40 megahertz (mHz) to about 3,000 megahertz (mHz). Therefore, because communication receiver 20 may be programmed to receive specific radio frequency ranges from transmitters 30, multiple receivers may be utilized in one central location, if need be. Communication receiver 20 includes one or more indicators. The indicators may be any type of indicator, including, but not limited to, a visual indicator, an audible indicator, etc. or any combination or derivation of these. Preferably the indicator is a visual indicator in the form of a display screen. Communication receiver 20 may include a separate indicator, or separate portions of one indicator, for each transmitter 30 which is programmed to transmit to communication receiver 20. Typically, up to about 25 (or more) transmitters 30 may transmit to one communication receiver 20.

Communication receiver 20 may be a multi-directional communication receiver. That is to say, communication receiver 20 receives the above-noted frequencies, or multiple frequencies, however, communication receiver 20 may also transmit radio frequencies within the ranges discussed above to transmitters 30. This allows a caregiver that is monitoring communication receiver 20 to shut off any local alarms (whether visual or audio) in a patient's room, from communication receiver 20. Additionally, the caregiver may reset the transmitter 30, adjust the programmed upper and lower limits of transmitter 30 and verbally communicate with the patient all from the caregiver's monitoring station.

Transmitter 30 communicates via a wireless communication system with communication receiver 20. Transmitter 30 is placed in close proximity to a patient, or alternatively, transmitter 30 is releasably attached directly to the patient. This may be done via any type of releasable connection including, but not limited to, VELCRO® straps, via a pouch or a sling worn by the patient, etc., or any combinations or derivations of these. This allows a patient to move throughout a hospital, a hospital system or outside of the hospital system while being monitored by a centrally located caregiver. That is to say, because transmitter 30 may be releasably connected to a patient, the patient may move about while still being monitored, so long as the patient does not move outside or beyond the wireless communication range of system 10.

The transmitter 30 contains known circuitry that is able to interpret a pulse or a pulse oximetry reading and the circuitry is further programmable to indicate when a patient's biological factors drop below a lower limit or exceed an upper limit. Transmitter 30 may emit a wireless communication frequency to communication receiver 20 when a patient's biological factors dip below a lower limit or exceed an upper limit, may emit a periodic wireless communication frequency to communication receiver 20 periodically (i.e., on a given time interval cycle—i.e., every 30 seconds) or may emit a constant wireless communication frequency to communication receiver 20 regardless of the patient's biological factors. Transmitter 30 may include a local indicator (i.e., an audio or visual alarm emitted by the transmitter). Transmitter 30 may also include an audible speaker and/or a microphone so that a centrally located caregiver and the patient can verbally communicate with each other.

Referring to FIG. 2, transmitter 30 may also search for an open frequency via known circuitry and programming technology. Communication receiver 20 is programmed to receive various wireless frequency ranges for each transmitter. For example, patients A, B and C are respectively given transmitter numbers #1, #2 and #3. When patient A's transmitter #1 transmits a wireless frequency to communication receiver 20, communication receiver 20 displays, or otherwise indicates, patient A's biological factors. Patient B's transmitter #2 then transmits a wireless frequency to communication receiver 20. Transmitter #2 is programmed to search for an open frequency range. Therefore, when transmitter #2 wirelessly communicates with communication receiver 20, communication receiver 20 then communicates with transmitter #2 indicating that the first receiver indicator is occupied by another transmitter (namely, transmitter #1 of patient A). Transmitter #2 then scans for the next open frequency. Communication receiver 20 may have up to 25 (or more) indicator fields, each indicator field has a separate wireless frequency range. Transmitter #2 then communicates patient B's biological factors to communication receiver 20 via the second open frequency. This is what is meant by transmitter 30 being able to scan for an open frequency. Transmitter 30 may be powered by any power source, however, a battery power source is preferred.

Transmitter 30 may optionally be sealed within any type of container. Typically, a hermetically sealed envelope or bag is preferred. This eliminates the need to sterilize the transmitters after each use.

Communication lead 40 includes a first end 41 releasably connected to transmitter 30 and a second end 42 connected to a reader 50. This connection is preferably a releasable connection. Reader 50 is adapted to read a variety of biological factors, namely the biological factors disclosed herein. Reader 50 utilizes known technology to read a variety of biological factors and may be placed anywhere on the body depending upon the biological factor being measured. This communication lead may be disposable.

Alternatively, reader 50 may not be connected to communication lead 40, but may be connected to, or include, a mini-transmitter. Reader 50 may then wirelessly transmit a patient's biological factors, via any of the various intervals provided above, either directly to transmitter 30 (see FIG. 3) or directly to communication receiver 20 (see FIG. 4). The mini-transmitter has incorporated into it all of the scanning and programmable features of the transmitter as discussed above. The mini-transmitter may also include an audible alarm. The audible alarm may be sounded when the patient's biological factors drop below a predetermined lower limit and/or exceed a predetermined upper limit.

In use, reader 50 periodically measures a patient's biological factors (i.e., pulse rate). This may be done by known detection methods using known technology. Such measurements may be taken, for example, about every 6 seconds, and then every other 6 second interval is compared to one another. Upon two or more compared 6 second intervals being elevated above an upper limit or being below a lower limit, an alarm and/or a communication sequence will follow to communication this to a centrally located caregiver. These timed intervals may vary according to the specific biologic factor one wants to measure. This helps to decrease and/or eliminate the majority of artifact.

In the foregoing description, it will be readily appreciated by those skilled in the art that modifications may be made to the invention without departing from the concepts disclosed herein. Such modifications are to be considered as included in the following claims, unless these claims by their language expressly state otherwise.

1. A patient monitoring system, comprising:
a stationary wireless communication receiver, wherein the receiver receives one or more wireless communication frequency and wherein the receiver includes at least one indicator;
at least one transmission device, which is in communication with a patient and is in wireless communication with the receiver, wherein the transmission device comprises a scanning and programmable transmitter which interprets and wirelessly communicates a patient's biological factors to the receiver via an open wireless communication frequency; and
a communication lead having a first end releasably connected to the transmitter and a second end in communication with a patient, which is adapted to interpret a patient's biological factors and wherein the biological factors are communicated via the communication lead to the transmitter.

2. The patient monitoring system of claim 1, wherein the receiver is in communication with at least one transmission device via wireless communication selected from the group consisting of uni-directional communication, bi-directional communication, multi-directional communication and any combination of these.

3. The patient monitoring system of claim 2, wherein the indicator comprises a display screen.

4. The patient monitoring system of claim 3, wherein the transmitter is programmable to alarm and wirelessly transmit information when a patient's biological factors exceed a predetermined upper limit and/or a predetermined lower limit.

5. The patient monitoring system of claim 4, wherein the transmitter transmits wireless communication to the receiver via a transmission selected from the group consisting of:
continuous transmission; periodic transmission; transmission that is activated when a patient's biological factors drop below a predetermined lower limit; transmission that is activated when a patient's biological factors exceed a predetermined upper limit and any combination of these.

6. The patient monitoring system of claim 5, wherein the biological factor comprises heart rate.

7. The patient monitoring system of claim 6, wherein the indicator comprises an audible alarm.

8. A patient monitoring system, comprising:
a stationary wireless communication receiver, wherein the receiver receives one or more wireless communication frequency and wherein the receiver includes at least one indicator;
a reader including at least one transmission device, which is in communication with a patient and is in wireless communication with the receiver, wherein the reader comprises a scanning and programmable microtransmitter which interprets and wirelessly communicates a patient's biological factors to the receiver via an open wireless communication frequency, wherein the reader is attached to a patient and adapted to interpret a patient's biological factors, which are then communicated to the transmission device and subsequently wirelessly communicated to the receiver.

9. The patient monitoring system of claim 8, wherein the receiver is in communication with at least one reader via wireless communication selected from the group consisting of uni-directional communication, bi-directional communication, multi-directional communication and any combination of these.

10. The patient monitoring system of claim 9, wherein the indicator comprises a display screen.

11. The patient monitoring system of claim 10, wherein the transmitter is programmable to alarm and wirelessly transmit information when a patient's biological factors exceed a predetermined upper limit and/or a predetermined lower limit.

12. The patient monitoring system of claim 11, wherein the microtransmitter transmits wireless communication to the receiver via a transmission selected from the group consisting of:
continuous transmission; periodic transmission; transmission that is activated when a patient's biological factors drop below a predetermined lower limit; transmission that is activated when a patient's biological factors exceed a predetermined upper limit and any combination of these.

13. The patient monitoring system of claim 12, wherein the biological factor comprises heart rate.

14. The patient monitoring system of claim 9, wherein the indicator comprises an audible alarm.
